(19) United States
(12) Patent Application Publication
(10) Pub. No.: US 2007/0029195 A1
(43) Pub. Date: Feb. 8, 2007

(54) Polymer/nanoparticle composites, film and molecular detection device

(76) Inventors: Changming Li, Singapore (SG); Wei Chen, Singapore (SG)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 11/196,792

(22) Filed: Aug. 3, 2005

(51) Int. Cl. G01N 33/487; G01N 31/00

(52) U.S. Cl. 204/403.01

(57) ABSTRACT

A molecular detection device for use in electrochemical detection assays includes at least two electrodes, and has a film deposited on at least one of the electrodes. The film includes a conductive polymer and conductive particles, having mean diameters between 1 and 100 nm, within the conductive polymer. Probe molecules may be attached on or to the conductive polymer, or be included in the conductive polymer. The device may be used to detect specific target molecules in a sample, for example, protein, peptide, nucleic acid or small molecule target molecules.

### FIELD OF THE INVENTION

The present invention relates to electrical or electrochemical detection devices, and more particularly to a polymer composite for use in such a device.

### BACKGROUND OF THE INVENTION

Electrical detection is based on the detection of alterations in the electrical properties of an electrode arising from interactions between probe and target molecules present in a reaction mixture. A device for electrically detecting biomolecules generally includes a supporting matrix on or in which to immobilize probe molecules. A solution, possibly containing target molecules, is placed in contact with the matrix having immobilized probe molecules, and changes in electrical properties are assessed.

Electrical detection eliminates many of the disadvantages inherent in use of radioactive or fluorescent labels to detect interactions between the probe and target molecules. For example, electrical detection is generally safe, inexpensive, and sensitive, and is not burdened with complex and onerous regulatory requirements.

Often, conductive polymers are used as the supporting matrix in electrochemical biosensors and bioelectronic devices. Such polymers are advantageous as they provide a matrix with a significant surface area for the relatively easy attachment of probe molecules. This in turn, yields a high concentration of probe molecules. Consequently, suitable polymers have been the subject of ever-increasing research efforts over the last few decades. For example, a glucose-oxidase enzyme, entrapped in the growing film of a polymer on the electrode using electrochemical methods, has been widely used to build a glucose sensor as, for example, detailed in S. Cosnier et al., J. Electroanal. Chem. 328, 361 (1992); M. Umana et al., Anal. Chem. 58, 2979 (1986); P. N. Bartlett et al., J. Electroanal. Chem. 224, 37(1987); N.C. Foulds et al., Anal. Chem. 60, 2473 (1988); D. Belanger et al., J. Electroanal. Chem. 274, 143 (1989); P. Janda et al., J. Electroanal. Chem. 300, 119 (1991); Y. Kajiya et al., J. Electroanal. Chem. 301, 155 (1991); M. Gao et al., Synth. Met. 137, 1393 (2003).

PCT patent publication WO 93/06237 similarly discloses chemical and biosensor devices based on electrochemically active polymer such as polypyrrole and polyaniline. Particularly, conductive polymer based electronic biosensors have been used in detection of DNA, peptides, and proteins, and such biosensors play important roles in characterizing the genome and proteome. For example, Lavache et al., Analytical Biochemistry 258, 188 (1998), describes an oligonucleotide array constructed on a silicon chip with a matrix of addressable microelectrodes. Each electrode is coated with polypyrrole containing functional groups to bind an oligonucleotide. Hepatitis C genotypes were detected by DNA hybridization using a fluorescent reporter molecule. Li et al., Frontiers in Bioscience, 10, 180-186, (2005), discloses a polypyrrole-based DNA biosensor with labelless detection based on the doping/undoping process of the polypyrrole.

Known detection devices use conductive polymers such as polypyrrole. However, obstacles in development of polymer matrices for detecting molecular interactions come from the degradation of the polymer when used in an electrical or electrochemical environment as, for example, detailed in J. Chem. Soc. 82, 1259, 1986; Li C. M. et al, Surface and Coatings Technology, 198(1-3), 2005. This is a particularly important consideration for making practical devices. Additionally, the sensitivity of conductive polymer-based biosensors is still in the range of μM to nM range. This is not sensitive enough to be used in medical diagnostic applications, especially for early diagnosis purposes.

As a result, there remains a need in the art to develop robust polymer matrices stable in the electrical or electrochemical devices for detecting interactions between biological molecules with high sensitivity and superior stability. The development of such devices would have wide application in the medical, genetic, and molecular biological arts.

### SUMMARY OF THE INVENTION

In one aspect of the present invention, there is provided a device for sensing the presence of specific target molecules, including a base; at least two electrodes formed on the base; and a film formed on a surface of at least one of the two electrodes. The film includes a conductive polymer and conductive particles having a mean diameter of between 0.1 nm and 100 nm.

In another aspect of the present invention, there is provided a polymer/particle composite including a conductive polymer matrix; and conductive particles having a mean diameter of between 0.1 nm and 100 nm within the polymer matrix.

In a further aspect of the invention, there is provided a method of forming a device for sensing the presence of specific target molecules, including forming at least two electrodes on a base; and forming a film including a conductive polymer and conductive particles having a mean diameter of between 0.1 and 100 nm on a surface of at least one of the two electrodes.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate by way of example only, embodiments of the present invention,

FIG. 1A is a top plan view of a molecular detection device, exemplary of an embodiment of the present invention;

FIG. 1B is a cross-sectional view of the device of FIG. 1A;

FIG. 2A is an Au 4f X-ray photon spectroscopy (“XPS”) spectrum of a polypyrrole/Au nanocomposite, exemplary of an embodiment of the present invention;

FIG. 2B is a scanning electron microscopy image of conventional polypyrrole;

FIG. 2C is a scanning electron microscopy image of a polypyrrole/Au nanocomposite, exemplary of an embodiment of the present invention;

FIG. 3 is a graph of impedance of a conventional device using pure polypyrrole film and an exemplary device using a polypyrrole/Au nanocomposite film;

FIG. 4 is a graph illustrating stability of conventional polypyrrole film and a polypyrrole/Au nanocomposite over time;

FIG. 5A is a graph of changes in electrode resistance for concentrations of anti-rat IgG in an exemplary device;

FIG. 5B is a graph of changes in electrode resistance for concentrations of anti-rabbit IgG;

FIG. 6 is a graph illustrating the stability of conventional polypyrrole and exemplary polypyrrole/carbon nanotube composite over time;

FIG. 7A is a graph of changes in resistance for concentrations of streptavidin in an exemplary device; and

FIG. 7B is a graph of changes in resistance for concentrations of rabbit IgG in an exemplary device;

### DETAILED DESCRIPTION

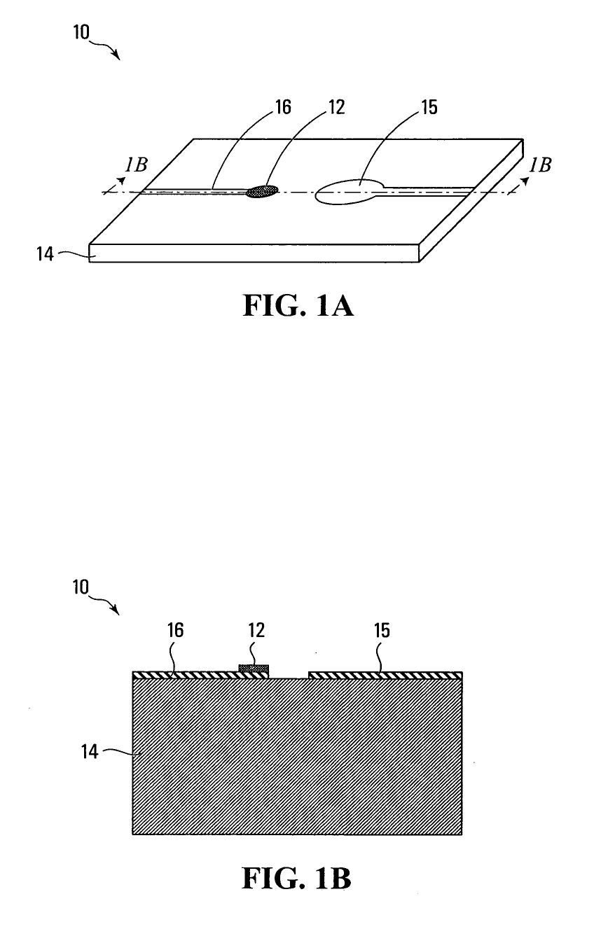

FIGS. 1A and 1B illustrate a molecular detection device 10, which includes a base 14 of a nonconductive material and at least one pair of electrodes 15, 16 extending on or to the surface of base 14. Electrodes 15, 16 are exemplified as counter and working electrodes, respectively. A polymer film 12 exemplary of an embodiment of the present invention is formed on top of the working electrode, 16. Example materials for base 14 include but are not limited to silicon, dioxide-topped silicon, ceramic, plastic, glass.

Polymer film 12 is electrochemically formed on the surface of working electrode 16. Specific binding molecules (often referred to as probe molecules) are immobilized on or within film 12. In general, the surface area of electrode 15 (counter) is larger than electrode 16 (working) by an order of magnitude. An electrolyte is applied between electrodes 15 and 16 and extends onto the electrode surface.

An electrical signal is applied at the pair of electrodes 15, 16. Changes in the electrical signal at electrode 16 are detected, in the presence of an electrolyte solution in contact with electrodes 15 and 16. Changes in the electrical characteristics of the electrodes 15, 16 may indicate the presence or absence of target molecules that bind to the probe molecules.

Many different geometries for detection device 10 are possible. Arrayed electrodes could be addressed for detection of multiple target molecules at different electrodes by multiplexing. Only one pair of electrodes, example working electrodes 16 and counter electrode 15, is depicted. However, any arbitrary number of electrodes could be formed on a suitable base 14. For example, electrodes may be arranged as counter electrodes in rows and working electrodes in columns. Current connectors may extend on one or both sides of base 14 and may be covered by insulation layer for exposing only the surface of the electrodes to the electrolyte. Other possible geometries for electrodes 15, 16 are described in PCT Application Nos. PCT/SG2005/000111; and PCT/SG2005/000112, the contents of which are hereby incorporated by reference. Yet others will be apparent to a person of ordinary skill.

Conventional conductive polymers, such as polypyrrole or polyaniline, used in the formation of electrochemical sensors are susceptible to degradation over time and also suffer from swelling. Such degradation typically begins with the nucleophilic attack of solution species on the polymer backbone bearing positive charges, as for example detailed in F. Beck et al., Ber Bunsenges Phys. Chem. 91, 967 (1987). The generation of positively charged centres in the polymer backbone like cation radicals (polarons) and especially dications (bipolarons) favours the nucleophilic attack of solution species. The solution attack causes a disruption of the conjugated network, a partial isolation of electronic communications between polymer molecules and a concomitant decrease in the contribution of intrachain charge transport to the total conductivity. The swelling process causes continuous changes of the bulk polymer structure resulting in conductivity changes. These problems have greatly impeded the use of known polymers in various novel applications.

Known preventative measures undertaken to inhibit the degradation process are collectively known as polymer stabilization. Because the oxidation potential of a conjugated polymer is normally lower than that of the monomer, the polymer may be oxidized during polymerization and counter-anions from the electrolyte are incorporated in order to maintain electrical neutrality. The nature of the incorporated counter-anion also determines the stability of the conductivity of the polymer. The size and shape of the counter-anions are important factors. The most stable polymer films are produced by the incorporation of small counter-anions. Presumably these counter-anions induce greater protection of the polymer chains against chemical attack by oxidants through increased oxidation and steric shielding. The incorporation of large counter-anions into polymer produce more unstable films, as detailed in B. R. Saunders et al., in Handbook of organic conductive molecules and polymers volume 3, Edited by H. S. Nalwa, John Wiley & Sons, 1997, p. 646.

Exemplary of embodiments of the present invention, polymer film 12 is formed as a polymer/particle composite and includes nanoparticles, promoting the stability of the film 12 and improving the sensitivity of the molecular detection device 10. Film 12 may, for example, be doped with such nanoparticles. The solid and rigid nanoparticles are entrapped in the polymer matrix, enforcing the polymer and significantly changing its physical bulk structure. The nanoparticles are embedded in the polymer such that large counter-anions could be effectively excluded from the conductive polymer and in the meantime the nanoparticles alleviate the polymer attack from the oxidant. Moreover, the nanoparticles create ion and electron conducting paths which improve the conductivity and rate of response performance of the conducting polymer in three ways. The first is by providing a large surface area of polymer in a porous morphology that enforces the structure of the polymer film, enhances adhesion and allows excellent electrolyte access in three dimensions. Second, since the polymer is coated on nanoparticles as a thin layer, the ion intercalation distance is reduced to a matter of nanometers. As well, the conductive nanoparticles dispersed throughout the structure increase the electrical conductivity of polymer film 12. Finally, the nanoparticles are rigid enough to enforce the polymer film 12 to prevent swelling that could result in significant change of the electric signal.

For example, and not by way of limitation, conductive polymers usable to form film 12 include polypyrrole, polythiophene, polyaniline, polyfuran, polypyridine, polycarbazole, polyphenylene, poly(phenylenevinylene), polyfluorene, polyindole, derivatives thereof, co-polymers thereof, and combinations thereof. Preferably the conductive polymer is polypyrrole, polythiophene and polyaniline, and most preferable is polypyrrole. As will be appreciated, a derivative of any of the exemplary conductive polymers includes the above mentioned conductive polymers having one or more substituents.

Suitable nanoparticles for polymer film 12 are conductive and include, but are not limited to, gold nanoparticles, platinum nanoparticles, carbon nanotubes, carbides, nitrides, fullerene, titanium oxide nanoparticles, zinc oxide nanoparticles, iron oxide nanoparticles, silicon nanoparticles, palladium nanoparticles, silver nanoparticles, copper nanoparticles, nickel nanoparticles, cobalt nanoparticles and combinations thereof. Other suitable nanoparticles will be appreciated by persons of ordinary skill, and are typically conductive with mean diameters between 1 and 100 nm (e.g. about 20 nm).

The concentration of the conductive nanoparticles in the matrix may be between 0.0001-1% w/w, or higher.

The exemplified conductive polymers are conjugated, and rely on delocation of π-electrons along the polymer backbone for conductivity. As noted, conventional cross-linked conjugated polymers trade-off conductivity for stability: the density of delocated π-electrons is inversely proportional to the stability of the polymer. Example polymers, however, are not cross-linked. For example, polypyrrole is a linear backboned polymer in one dimension.

Instead, example conductive nanoparticles insert or attach to the polymer backbone and can serve as electron mediators, greatly improving the polymer's conductivity without cross-linking. Additionally, the nano-particles form weak or strong bonds, which are typically non-covalent, with adjacent polymers in an array and thus strengthen the polymers.

It is believed that atoms on the nanoparticle surface, such as carbon, may co-ordinate with atoms in the polymer backbone, for example nitrogen, thereby forming a connection between polymer molecules without sacrificing the π-electron density.

In one embodiment, film 12 may thus take the form of a polymer/nanoparticle conductive matrix layer of polypyrrole/gold nanoparticle composite, or polypyrrole/carbon nanotubing composite.

Electrodes 15, 16 may be formed of a gold, platinum or glassy carbon conductor, solid or porous, foils or films of silver, titanium, or copper, or metal oxide, metal nitrides, metal carbides, carbon, graphite, or combinations thereof, or other materials appreciated by those of ordinary skill.

Use of conductive nanoparticles improves the stability of film 12 for biomolecule probe attachment or entrapment in detection device 10. Probe molecules may be attached using bioconjugation at one or more functional groups in each probe molecule, using a precursor solution containing regular monomer, unique monomer with functional group (“functionalized monomer”) and nanoparticles to copolymerize the composite thin film for probe biomolecule attachment, as shown in the examples. Probe molecules may, for example, be non-covalently entrapped within the polymer film 12, covalently embedded within the polymer matrix formed by film 12 or covalently attached to the surface of the polymer.

In non-covalent incorporation, the probe molecule may be mixed with a monomer followed by polymerization of the monomer, which immobilizes the probe molecule within the polymer matrix forming film 12.

Alternatively, the probe molecules may be covalently attached to the monomer. Polymerization of the monomer may then immobilize the probe molecule within the polymer matrix.

In another alternative, probe molecules may be non-covalently entrapped in or attached to the polymerized conductive polymer matrix or covalently attached to the polymer backbones by various linkers and corresponding functional groups. For example and without limitation, linkers to attach probe molecules to the surface of the conductive polymer or to the monomers of the conductive polymer include, without limitation, NHS-ester, maleimide, imidoester, active halogen, carboxylic acid-EDC, pyridyl disulfide, azidophenyl, vinyl-sulfone, hydrazide, isocyanate, biotin. The probe and target molecules may be a nucleic acid, DNA, RNA, protein or peptide (for example, an antibody or antibody fragment or an antigen), an aptomer or a small molecule. The probe molecule has specific binding affinity for the target molecule and will therefore specifically bind to the target molecule when probe molecule comes into contact with a solution containing target molecule. In a preferred embodiment the present invention does not use a label or a reporter group or molecule, electrochemical or otherwise, attached to probe or target molecule.

In one embodiment, a specific monomer pyrrole, pyrrole propylic acid, with functional group for covalently binding probe molecules, is designed and synthesized for highly efficient immobilization of probe molecules such as proteins onto polypyrrole backbones through chemical reaction of hydroxyl group and amine groups.

To form the polymer/nanoparticle film 12 on electrodes 15, 16 a precursor solution containing monomers, for covalent probe immobilization, and nanoparticles may be formed. The solution may then be electrochemically polymerized and deposited on an electrode surface in a single step to generate a polymer or copolymer for use in film 12. Conventional electrochemical polymerization methods include but are not limited to cyclic voltammetry, constant potential deposition, or constant current deposition. The thickness and porosity of film 12 can be controlled by concentration of the precursor monomer solution. They can also be controlled by scan rate, magnitude of potential, and magnitude of current density, respectively, in the three methods described above.

Alternatively, the polymer nanoparticle film 12 can be chemically formed on the surface of electrodes 15, 16 by addition of strong oxidants. A conjugated polymer or copolymer may be deposited in a charged, conductive state. The polymer or copolymer may then be electrochemically synthesized with conductive nanoparticles to form the polymer nanoparticle film 12.

Example polymers or copolymers with incorporated nanoparticles have low electric background when used in the electric detection of biomolecule. Conveniently, resulting device 10 may have significantly an improved signal to noise ratio, thus enhancing the sensitivity of biomolecule detection.

In yet another embodiment, device 10 further includes one or more additional reference electrodes. The counter-electrode includes a conductive material with an exposed surface that is significantly larger than that of the working electrodes, and a reference electrode is not needed for simple device fabrication. In one embodiment, the counter electrode comprises platinum foil. In alternative embodiments, as shown in FIG. 1A, the counter electrode comprises solid or porous films of gold, silver, platinum, titanium, or copper, or metal oxides, metal nitrides, metal carbides, carbon, graphite, or combinations thereof. The reference electrode may be formed as a silver/silver chloride or saturated calomel electrode.

Electrochemical contact between each of electrodes 15, 16 and/or the reference electrode is provided using an electrolyte solution or a solid or gel electrolyte in contact with each of the electrodes. Suitable electrolyte solutions include any electrolyte solution at physiologically-relevant ionic strength (equivalent to about 0.15 M NaCl) and neutral pH. Examples of electrolyte solution include, but are not limited to, phosphate buffered saline, HEPES buffered solution, and sodium bicarbonate buffered solutions. Example solutions do not disrupt or denature the probe and target molecules so as not to interfere with the probe/target molecule specific interaction. These electrolyte solutions are in contact with each of electrode 16 (i.e. the working electrode), the counter electrode 15 (i.e. the counter electrode) and the reference electrode if provided, thereby providing electrochemical contact between the electrodes.

Device 10 may be used for the electrical detection of the presence of a target molecule based upon a molecular interaction between a probe molecule and the target molecule. An electrical property of electrodes 15, 16 is measured, with film 12 having only probe molecules immobilized thereto. Next, film 12 is exposed to a sample mixture possibly containing the target molecule. The electrical property of electrodes 15, 16 is again measured. Before the second measurement, non-reacted target molecules may be removed by washing in order to reduce non-specific binding noise. The two measurements are compared to determine whether a molecular interaction between the probe and the target molecule occurred, which will confirm whether the target molecule is present in the sample mixture. The electrical property may be the impedance of electrodes 15, 16.

Electrical impedance may be measured using an impedance analyser with an electrochemical interface. Alternatively, transients could be measured using an AC signal perturbation superimposed on a DC potential applied to an electrochemical cell such as AC bridge and AC voltammetry. The measurements can be conducted at a certain particular frequency that specifically produces electrical signal changes that are readily detected or otherwise determined to be advantageous. Such particular frequencies are advantageously determined by scanning frequencies to ascertain the frequency producing, for example, the largest difference in electrical signal, in manners understood by those of ordinary skill. Impedance at each electrode as a result of, for example, antibody-antigen binding, or any other probe-target interaction may be measured using any of the above-described instruments and analytical methods, or others understood by persons of ordinary skill.

In order to reduce or eliminate variations from different single electrodes in multi-concentration analyses, relative changes in impedance may be measured. This measurement is dimensionless. For example, the resistances measured at a probe-impregnated electrode before and after the target incubation may be measured as R₁ and R₂, respectively. The dimensionless resistance unit change, ΔR_{N}, may then be calculated as

$$Δ {R}_{N} = \frac{{R}_{2} - {R}_{1}}{{R}_{1}}$$

ΔR_{N} represents the dimensionless unit resistance change. The normalized dimensionless unit resistance change is based on results from a single working electrode. In some cases, such as devices that are used as immunosensors, a single sensor cannot be used for multi-concentration analysis. When multiple electrodes are used for multiconcentration analysis, the dimensionless unit impedance change represents the changes per unit impedance, and use of this dimensionless measurement helps to eliminate the variation of thickness and surface area of the working electrodes. Thus, measurements even between different electrodes allows for quantification of the change resulting from the probe-target molecule interactions in the polymer matrix, rather than the change of the bulk electric properties of film 12, thus eliminating or reducing the variation of bulk resistance caused by variations of the polypyrrole films, particularly between different working electrodes. Device 10 can include a suitable electrical impedance device to measure the impedance and calculate dimensionless in impedance.

While not wishing to be bound by any particular theory, it is thought that the molecular interaction of a target molecule and a probe molecule immobilized in or on a conductive polymer matrix interferes with the ion interaction process resulting in an increase of the resistance. It has been found that the matrix resistance significantly increases at the testing site, as shown in the examples below, after probe/target molecular interaction, such as antibody/antigen bindings. The detection is accomplished without labeling the target or probe molecules: it is again noted that this preferred embodiment does not use an electrochemical, fluorescent, radioactive or other type of reporter attached to the target or probe molecules. This is quite attractive because it can significantly reduce the manufacturing cost and simplifies the detection process.

Application areas for the exemplary film 12 and device 10 include diagnostics, therapeutics, pre-clinical and clinical trials, target discovery, target validation, pathogen detection for drug discovery, health care, food processing, environmental monitoring, and defense.

Particularly, aspects of device 10 provide a basic platform for the electrical or electrochemical detection of biomolecules. For example, embodiments of this invention can be used to make a protein biosensor for an immunoassay which can provide an extremely high-sensitivity method for clinical laboratory diagnosis.

Aspects exemplary of embodiments of the invention will be further described by the following example and figures with polypyrrole/nanoparticle composite as the matrix and protein as detection target. The examples are intended to illustrate specific embodiments, but not to limit the scope of the invention.

### EXAMPLE 1

3.8 mL pyrrole (55 mmol) and 0.3 mL benzyltrimethylammonium hydroxide were added to a 50 mL flask. To this solution, 2.9 mL acrylonitrile (55 mmol) was added gradually. The addition was controlled so the temperature of the mixture did not exceed 40° C., to prevent a strong exothermic reaction. The mixture was stirred at room temperature overnight.

The mixture was hydrolyzed by addition of 50 mL 10 N potassium hydroxide solution. The aqueous solution was refluxed overnight. After the solution cooled down, HCl was gradually added to acidify the solution till pH reached 3 on pH paper. The aqueous layer was extracted with ethyl acetate four times, each time using 30 mL solvent. The combined organic layer was washed with 75 mL brine, then dried with anhydrous magnesium sulfate. The solvent was evaporated under rotavap and 6 g brown solid power was obtained. The crude product was crystallized by methylene chloride and hexane. 5 g pure pyrrole propylic acid was obtained (65% yield). ¹NMR (CDCl₃): δ2.83 ppm (t, 2H, J=7.2 Hz), δ4.20 ppm (t, 2H, J=7.2 Hz), δ6.14 ppm (t, 2H, J=2, 1 Hz), δ6.67 ppm (t, 2H, J=7.2 Hz), δ9.00 ppm (s, broad peak 1H),

The reaction may be described as,

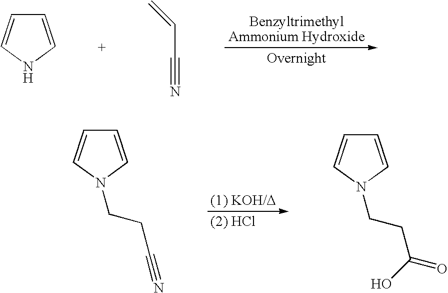

This compound allows for covalent attachment of probe molecules such as proteins onto polypyrrole backbones through chemical reaction of hydroxyl group and amine groups.

### EXAMPLE 2

Gold nanoparticles were prepared by modified tannic acid/citrate method as for example described in J. W. Slot et al., Eur. J. Cell. Biol. 38, 87 (1985). Specifically, two solutions were used: (a) the Au³⁺ solution, containing 1 mL of 1% HAuCl₄ in 79 mL deionized water; and (b) the reducing mixture, consisting of 155 μL of 1M tri-sodium citrate, 4 mL of 1% tannic acid, 0.2 mL of 0.1 M K₂CO₃ and deionized water to bring the total volume of (b) up to 20 mL. Both (a) and (b) were brought to 60° C. on a hot plate. Then the reducing mixture (b) was quickly added to the Au³⁺ solution (a) while stirring. Finally, the solution was heated until boiling. The prepared gold nanoparticle (˜3 nm diameter) solution was further concentrated by evaporating the solution to 50 mL and the final concentration of the gold nanoparticle was about 0.88 mM.

### EXAMPLE 3

Thereafter, a solution containing 0.4 M pyrrole (Aldrich), 0.01 M PBS buffer and 0.88 mM Au nanoparticle was prepared. A film formed of polypyrrole/Au nanocomposite layer was synthesized by an electrochemical method. Glassy carbon was used as the working electrode. A platinum foil, with much larger surface area than that of the working electrode, and an Ag/AgCl electrode was used as counter and reference electrodes, respectively. An EG&G 273A potentiostat/galvanostat was employed for the synthesis of the polypyrrole/Au nanocomposite films onto the surface of working electrode by applying by 1.5 mAcm⁻² constant current for 1800 s. After the deposition, a Solartron 1260 impedance freqency analyzer coupled with a Solartron 1287 electrochemical interface was used to measure the impedance of the working electrode in PBS buffer.

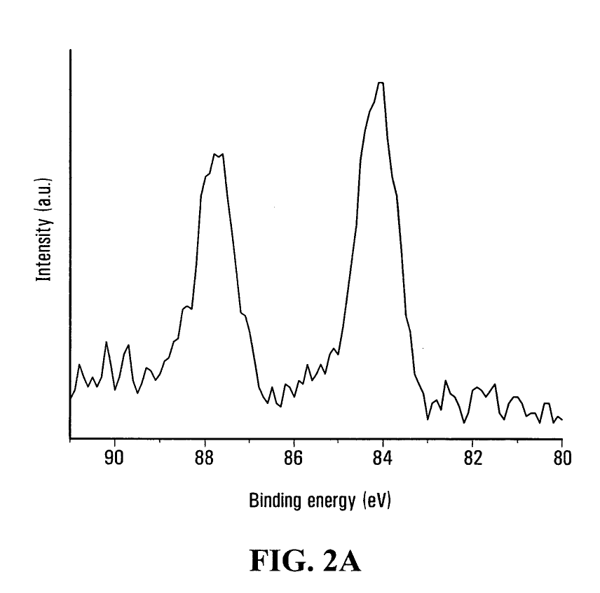

FIG. 2A illustrates the Au 4f XPS spectrum of the polypyrrole/Au nanocomposite showing the existence of the Au in the polypyrrole/Au nanocomposite and elements concentration in the nanocomposite film is given in Table 1, in which the atomic concentration of Au in polypyrrole/Au nanocomposite is about 0.12%.

TABLE 1
Elements concentration in the polypyrrole/Au nanocomposite film

| | O | N | C | Au |
|---|---|---|---|---|
| Atomic Concentration % | 21.89 | 8.46 | 69.54 | 0.12 |
| Mass Concentration % | 26.39 | 8.93 | 62.96 | 1.72 |

FIGS. 2B and 2C show the surface morphologies of polypyrrole and polypyrrole/Au nanocomposite taken by scanning electron microscopy. The morphologies in FIG. 2B are typical of those reported for polypyrrole films, showing clusters of small overlapping hemispheres, as for example described in R. Qian et al., Synth. Met. 18, 13 (1987); D. S. Maddison et al., Synth. Met. 30, 47 (1989). The Au nanoparticle has affected the morphology and increased the surface area of polypyrrole as shown in FIG. 2C in which the hemispheres appear more fibrous than the one in FIG. 2B.

Three-electrode measurement was used. The counter electrode was platinum foil, the surface area of which was much larger than the working electrode and the reference electrode was Ag/AgCl electrode. To provide a basis for comparison, these tests were also performed on pure polypyrrole films made using the same setup and conditions without the Au nanoparticle in the aqueous polymerization electrolyte.

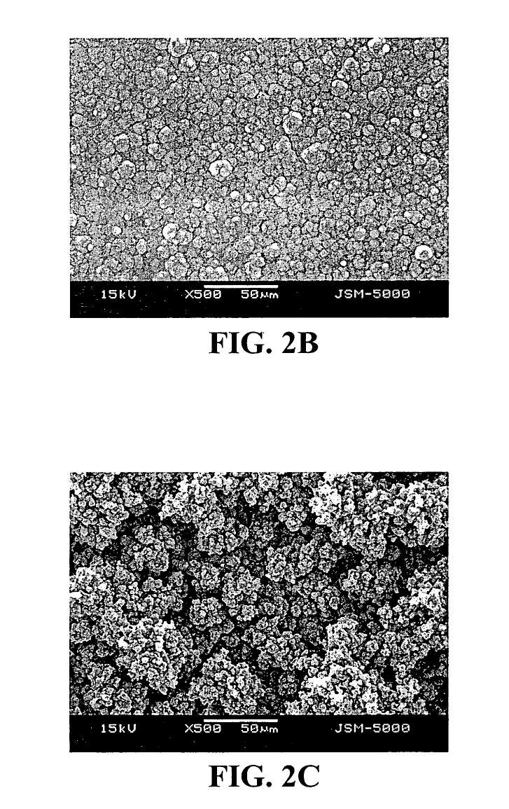

FIG. 3 is a plot of the electrochemical impedance spectra which demonstrate the difference in conductive behavior between pure polypyrrole and polypyrrole/Au nanocomposite films in 0.01 M PBS buffer. In comparison to similarly prepared pure polypyrrole films, exemplary nanocomposite films mainly exhibit diffusive behavior, a result that can only be attributed to the presence of Au nanoparticle within the nanocomposite films. The intercept with the real impedance (Z′) axis of these plots indicates the combined uncompensated electrical resistance of the film, electrolyte, and the electrical leads. Assuming the difference in electrical resistance of the electrolyte and leads to be negligible with respect to that of the electrochemically active films, the lower real axis intercept of the nanocomposite film relative to the pure polypyrrole films is indicative of a conductive contribution from the Au nanoparticle. Also it should be noted that the reduced resistance of the nanocomposite film may be partially due to the increased surface area of the nanocomposite structure.

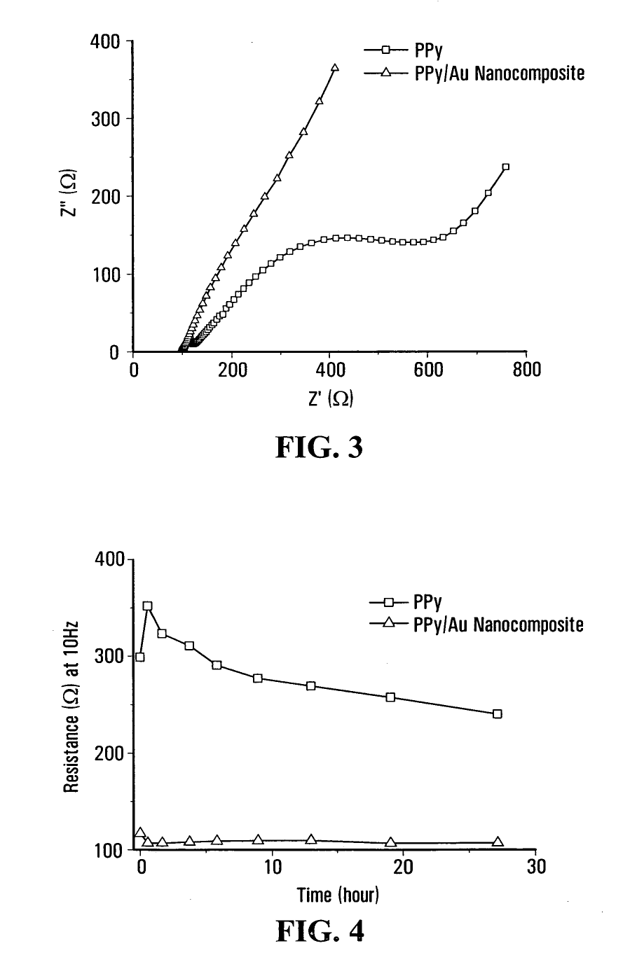

Polypyrrole and polypyrrole/Au nanocomposite film were prepared and tested by using the electrochemical procedure described in this Example, respectively. Their stabilities in PBS buffer were investigated by measuring their impedance variation at 10 Hz for 0, 0.5, 1.5, 4, 6, 9, 13, 19, 27 hours, respectively after deposition, as shown in FIG. 4. It was observed the nanocomposite film has lower and more stabilized resistance as compared to the pure polypyrrole film, which shows that Au nanoparticle dispersed throughout the structure not only increases the electrical conductivity but also improve the stability of polypyrrole film.

### EXAMPLE 4

Gold nanoparticles were prepared by the modified tannic acid/citrate method described above. A solution containing 0.26M pyrrole, 0.065M pyrrole propylic acid (PPA), 0.15 mM Au nanoparticle and PBS buffer was prepared for the electrochemical deposition of polypyrrole/PPA/Au nanocomposite film on the glassy carbon electrode, as described above. After deposition, the film was soaked in 1.5% EDC in acetonitrile for 1.5 hours to activate the carboxylic group in PPA. An 8 μL of 1 mg/mL streptavidin as probe was added onto the nanocomposite film. After 12 hours incubation, the working electrodes were rinsed in PBS solution for 1 hour and then dried. After the probe molecules deposited on the working electrodes surface, AC impedance was measured. After a baseline reading, 0, 10 fg/mL, 100 fg/mL, 1 pg/mL, and 10 pg/mL anti-streptavidin in PBS solution were prepared. The glassy carbon electrodes were incubated in these solutions for 2.5 hours at room temperature. The electrodes were then rinsed vigorously in a PBS solution and dried. Impedance measurements were taken of each electrode again. The change in resistance for each electrode before and after incubation was thus obtained.

### EXAMPLE 5

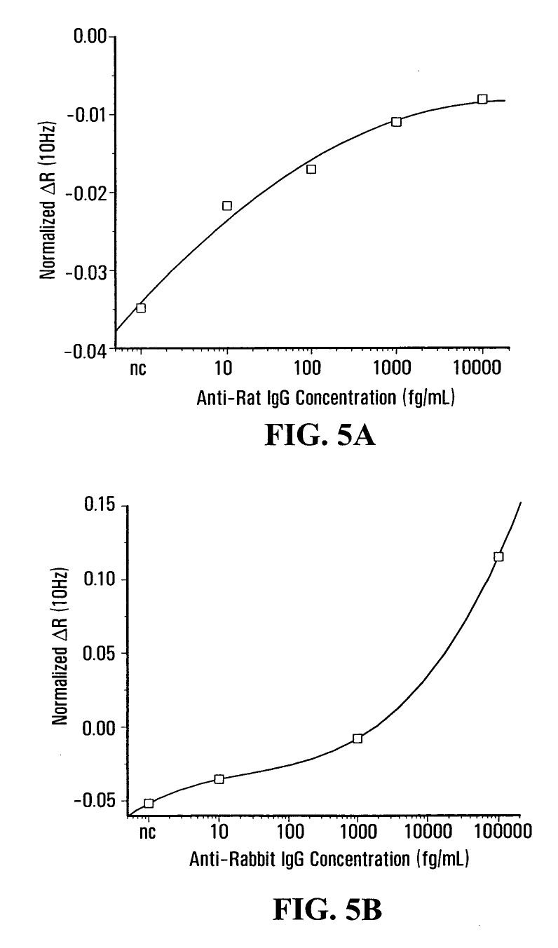

1 mg/mL rat IgG as probe molecule in PBS solution was immobilized on the glassy carbon electrodes by immobilization on the polypyrrole/PPA/Au nanocomposite film using the procedure described in EXAMPLE 3. AC impedance was measured to obtain a baseline reading. The electrodes were then incubated in solutions of 0, 10 fg/mL, 100 fg/mL, 1 pg/mL, and 10 pg/mL anti-rat IgG for 2.5 hours, respectively. The electrodes were rinsed vigorously in a PBS solution and dried. Impedance measurements were taken of each electrode again. FIG. 5A charts the dimensionless resistances calculated based on measured resistances at 10 Hz in a buffer solution before after incubation of a rat IgG attached electrode in solutions containing different concentrations of the target molecule, anti-rat IgG. These results further demonstrate the high sensitivity down to at least 10 fg/mL for detecting the target can be obtained using polymer/nanoparticle composite supporting matrix.

### EXAMPLE 6

1 mg/mL rabbit IgG as probe molecules in PBS solution had been immobilized in the glassy carbon electrodes by the polypyrrole/PPA/Au nanocomposite film using the procedure described in Example 3. After a baseline reading, the electrodes were incubated in solutions of 0, 10 fg/mL, 1 pg/mL, and 100 pg/mL anti-rabbit IgG for 2.5 hours, respectively. The electrodes were rinsed vigorously in a PBS solution and dried. Impedance measurements were taken of each electrode again. The average of the change in resistance at 10 Hz for each of the solutions is plotted versus the concentration of each of the solutions in FIG. 5B. These results further confirm that nanoparticles included in the polymer film can improve the detection sensitivity on targets.

### EXAMPLE 7

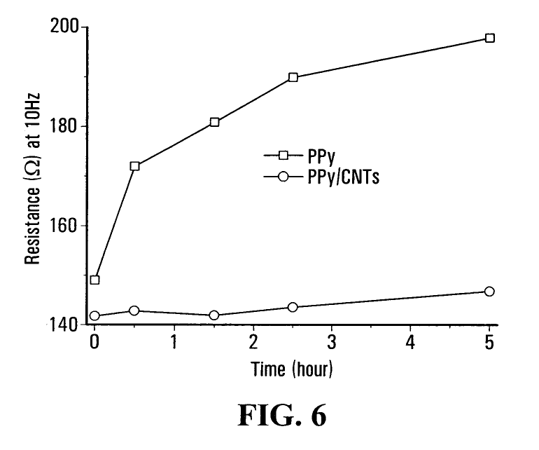

A solution containing 0.4 M pyrrole, 0.01 M PBS buffer and 0.005% single wall carbon nanotubes (CNTs, ˜1 nm, Aldrich) was prepared. In order to obtain a uniform polypyrrole coating, CNTs were pretested in 15 wt % HNO₃ aqueous solution to increase the electrochemical activity of the nanotube surface. The polypyrrole/CNTs nanocomposite layer was electrochemically synthesized on the surface of gold working electrode by applying by 3.33 mAcm⁻² constant current for 300 s. The electrochemical synthesis was as described in example 1. After the deposition, AC impedance was measured on gold working electrode with the same procedure described in EXAMPLE 1. To provide a basis for comparison, these tests were also performed on pure polypyrrole films made using the same setup and conditions without the carbon nanotubes in the aqueous polymerization electrolyte. The stabilities of electrodes in PBS buffer were investigated by measuring their impedance variation at 0, 0.5, 1.5, 2.5, and 5 hours after the polymer deposition, as shown in FIG. 6. It has been observed the nanocomposite film has lower and more stabilized resistance as compared to the pure polypyrrole film, which shows that CNTs dispersed throughout the structure not only increases the electrical conductivity but also improve the stability of polypyrrole film.

### EXAMPLE 8

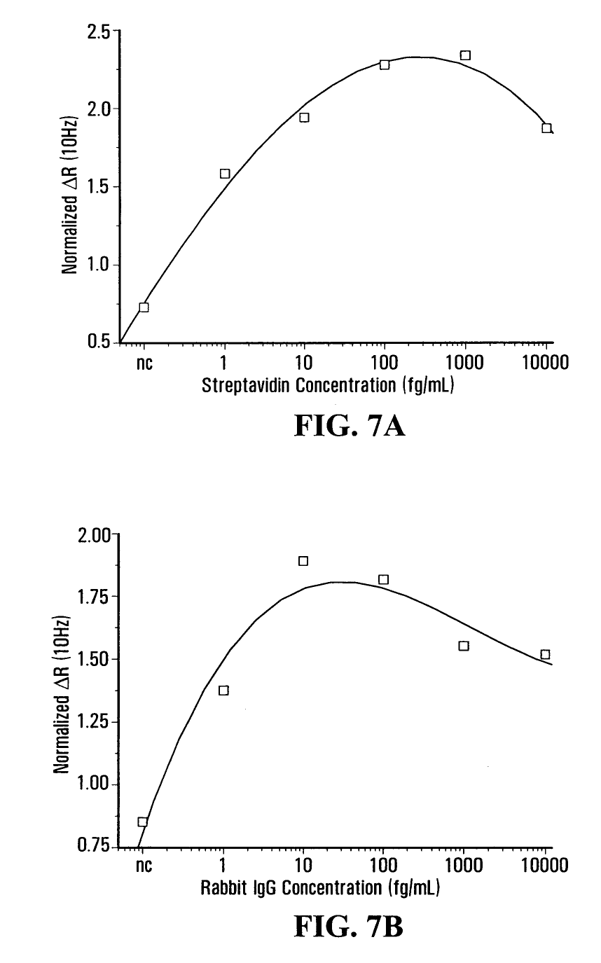

A solution containing 0.4 M pyrrole, 0.01 M PBS buffer and 0.005% single wall carbon nanotubes was prepared as solution M. A solution for probe synthesis was prepared by adding anti-streptavidin as probe into the solution M and the final concentration of the probe was 200 ug/mL. The polypyrrole/CNTs/probes layer was synthesis by an electrochemical method described in EXAMPLE 4. After rinsing the nanocomposite layer with PBS solution, AC impedance was measured with the same procedure described in Example 6. After a baseline reading, the gold electrodes were incubated in solutions of 1 fg/ml, 10 fg/ml, 100 fg/ml, 1 pg/ml, and 10 pg/ml streptavidin for 2.5 hours, respectively. The electrodes were then rinsed vigorously in a PBS solution and dried. Impedance measurements were taken of each electrode again. The dimensionless impedances were calculated based on measured resistances at 10 Hz in a buffer solution before after incubation of an anti-streptavidin-attached electrode in solutions containing different concentrations of the target molecule, streptavidin. As illustrated in FIG. 7A, the average of change in dimensionless resistance at 10 Hz for each of the solutions is plotted versus the concentrations of each of the solutions. These results demonstrate that the present method in which the probe molecules are non-covalently immobilized within the nanoparticle-incorporated polymer matrix for electrical detection can detect the presence of target analyte down to at least 1 fg/mL, with a dynamic range of over three-orders of magnitude to reach a plateau response.

### EXAMPLE 9

A solution containing 0.4 M pyrrole, 0.01 M PBS buffer and 0.005% single wall carbon nanotubes, and 200 ug/mL anti-rabbit IgG was prepared for probe synthesis. Polypyrrole/CNT/probe films were electrochemically deposited on gold working electrodes using the procedure described in Example 6. After a baseline reading, the gold electrodes were incubated in solutions of 1 fg/ml, 10 fg/ml, 100 fg/ml, 1 pg/ml, and 10 pg/ml rabbit IgG for 2.5 hours, respectively. The electrodes were rinsed vigorously in a PBS solution and dried. Impedance measurements were taken of each electrode again. FIG. 7B is a graph of the average of the change in dimensionless resistance at 10 Hz for each of the solutions is plotted versus the concentration of each of the solutions. These results further confirm that nanoparticle can improve the detection sensitivity on targets.

Of course, the above described embodiments and examples are intended to be illustrative only and in no way limiting. The described embodiments of carrying out the invention are susceptible to many modifications of form, arrangement of parts, details and order of operation. The invention, rather, is intended to encompass all such modification within its scope, as defined by the claims.

What is claimed is:

1. A device for sensing the presence of specific target molecules, comprising
a base;
at least two electrodes formed on said base;
a film formed on a surface of at least one of said two electrodes;
said film comprising a conductive polymer and conductive particles having a mean diameter of between 0.1 nm and 100 nm.

2. The device of claim 1, wherein said film comprises a polymer matrix, and wherein said conductive particles are embedded therein.

3. The device of claim 1 wherein said film is electrochemically deposited onto said at least one of said two electrodes from a precursor solution.

4. The device of claim 2, wherein said polymer comprises at least one of polypyrrole, polythiophene, polyaniline, polyfuran, polypyridine, polycarbazole, polyphenylene, poly(phenylenevinylene), polyfluorene and polyindole, or derivatives thereof, or co-polymers thereof.

5. The device of claim 2, wherein said base is formed of at least one of silicon dioxide-covered silicon, ceramic, glass, and plastic.

6. The device of claim 5, further comprising probe molecules attached on or within said film.

7. The device of claim 6, wherein said probe molecules are non-covalently entrapped within said film.

8. The device of claim 6, wherein said probe molecules are covalently embedded in said film.

9. The device of claim 6, wherein said probe molecules are covalently attached to the surface of said conductive polymer by linkers.

10. The device of claim 9, wherein said linkers comprise NHS-ester, maleimide, imidoester, active halogen, carboxylic acid-EDC, pyridyl disulfide, azidophenyl, vinyl-sulfone, hydrazide, or isocyanate.

11. The device of claim 1, wherein one of said two electrodes is formed of at least one of gold, platinum, glassy carbon, silver, titanium, copper, metal oxide, metal nitrides, metal carbides, carbon and graphite.

12. The device of claim 2, wherein said conductive particles comprise at least one of gold nanoparticles, platinum nanoparticles, carbon nanotubes, fullerene, titanium oxide nanoparticles, zinc oxide nanoparticles, iron oxide nanoparticles, metal carbide nanoparticles, metal nitride nanoparticles, silicon nanoparticles, palladium nanoparticles, silver nanoparticles, copper nanoparticles, nickel nanoparticles and cobalt nanoparticles.

13. The device of claim 1, wherein one of said two electrodes is a counter electrode formed of material selected from gold, silver, platinum, titanium, copper, metal oxides, metal nitrides, metal carbides, carbon and graphite, or combinations thereof.

14. The device of claim 1, further comprising at least one reference electrode formed of material selected from silver/silver chloride and saturated calomel.

15. The device of claim 1, wherein said conductive polymer is polypyrrole.

16. The device of claim 3, wherein said precursor solution contains at least one of pyrrole, carbon nanotubes, gold nanotubes, and pyrrole propylic acid.

17. The device of claim 1, further comprising an electrical impedance measuring device to measure electrical impedance between said two electrodes.

18. The device of claim 17, wherein said impedance measuring device determines dimensionless changes in impedance before and after the target incubation.

19. A polymer/particle composite comprising:
a conductive polymer matrix;
conductive particles having a mean diameter of between 0.1 nm and 100 nm within said polymer matrix.

20. The polymer/particle composite of claim 19, wherein the concentration of said conductive particles in said matrix is between 0.0001-1%.

21. The polymer/particle composite of claim 20, wherein said polymer matrix comprises at least one of polypyrrole, polythiophene, polyaniline, polyfuran, polypyridine, polycarbazole, polyphenylene, poly(phenylenevinylene), polyfluorene and polyindole, or derivatives thereof, or co-polymers thereof.

22. The polymer/particle composite of claim 21, further comprising probe molecules immobilized on or within said conductive polymer matrix.

23. The polymer/particle composite of claim 22, wherein said probe molecules are non-covalently entrapped within said conductive polymer matrix.

24. The polymer/particle composite of claim 22, wherein said probe molecules are covalently embedded within said conductive polymer matrix.

25. The polymer/particle composite of claim 24, wherein said probe molecules are covalently attached to the surface of said conductive polymer matrix.

26. The polymer/particle composite of claim 24, wherein said probe molecules are covalently attached to the surface of said conductive polymer matrix by linkers.

27. The polymer/particle composite of claim 24, wherein said probe molecules comprise a nucleic acid molecule, a DNA molecule, an RNA molecule, a protein, a peptide, a small molecule or an aptomer.

28. The polymer/particle composite of claim 26, wherein said linkers comprise at least one of NHS-ester, maleimide, imidoester, active halogen, carboxylic acid-EDC, pyridyl disulfide, azidophenyl, vinyl-sulfone, hydrazide, and isocyanate.

29. The polymer/particle composite of claim 19, wherein said conductive particles comprise at least one of gold nanoparticles, platinum nanoparticles, carbon nanotubes, fullerene, titanium oxide nanoparticles, zinc oxide nanoparticles, iron oxide nanoparticle, silicon nanoparticles, palladium nanoparticles, silver nanoparticles, copper nanoparticles, nickel nanoparticles and cobalt nanoparticles.

30. A method of forming a device for sensing the presence of specific target molecules, comprising:
forming at least two electrodes on a base;
forming a film comprising a conductive polymer and conductive particles having a mean diameter of between 0.1 and 100 nm on a surface of at least one of said two electrodes.

31. The method of claim 30, further comprising immobilizing probe molecules on or within said film.

32. The method of claim 30, wherein said conductive particles comprise at least one of gold nanoparticles, platinum nanoparticles, carbon nanotubes, fullerene, titanium oxide nanoparticles, zinc oxide nanoparticles, iron oxide nanoparticle, silicon nanoparticles, palladium nanoparticles, silver nanoparticles, copper nanoparticles, nickel nanoparticles and cobalt nanoparticles.

33. The method of claim 32, wherein said forming said film comprises forming a precursor solution and electrochemically depositing said precursor solution onto said at least one of said two electrodes.

34. The method of claim 33, wherein said precursor solution comprises a monomer and conductive nanoparticles.

35. The method of claim 33, wherein said precursor solution comprises a regular monomer, a functionalized monomer and conductive nanoparticles.

36. The method of claim 33, wherein said precursor solution contains at least one of pyrrole, carbon nanotubes, gold nanotubes, and pyrrole propylic acid.

37. The method of claim 33, wherein said electrochemical depositing comprises using cyclo-voltammetry.

38. The method of claim 33, wherein said electrochemical depositing comprises electrochemical deposition under a constant potential.

39. The method of claim 33, wherein said electrochemical depositing comprises electrochemical deposition under a constant current.
